(19) United States
(12) Patent Application Publication
(10) Pub. No.: US 2024/0342155 A1
(43) Pub. Date: Oct. 17, 2024

(54) HYDROXYCHLOROQUINE FORMULATIONS, DOSAGE FORMS, AND METHODS OF USE

(71) Applicant: SLBST PHARMA, INC., Tampa, FL (US)

(72) Inventors: Beat WEIDMAN, Chatham, NJ (US); Dan H. KIM, Morris Plains, NJ (US); Mark CONNELL, Chestnut Hill, MA (US); John CARTER, Tampa, FL (US)

(21) Appl. No.: 18/580,371

(22) PCT Filed: Jul. 21, 2022
(86) PCT No.: PCT/US2022/037924

§ 371 Date: Jan. 18, 2024

Related U.S. Application Data
(60) Provisional application No. 63/224,179, filed on Jul. 21, 2021.

(51) Int. Cl. A61K 31/4706; A61K 9/20; A61K 9/28; A61K 45/06

(52) U.S. Cl. CPC A61K 31/4706; A61K 9/2068; A61K 9/28; A61K 45/06

(57) ABSTRACT

Pharmaceutical compositions and formulations containing hydroxychloroquine having improved safety and tolerability are disclosed herein. In addition, methods of treating disease conditions, including autoimmune diseases, retinopathy and endometriosis. with such compositions and formulations are disclosed.

### FIELD

This application relates generally to formulations and dosage forms of hydroxychloroquine and their use in treating disease with improved safety and tolerability.

### BACKGROUND

Hydroxychloroquine (HCQ), an hydroxylated, less toxic derivative of chloroquine, is a synthetic drug initially developed in the 1950s for the prevention and treatment of malaria. HCQ was FDA approved in 1955 for this indication, as the New Molecular Entity branded Plaquenil®. As an antimalarial drug, HCQ works by directly killing the organism that causes malaria. Today, however, HCQ is mainly used as disease-modifying drug for autoimmune diseases like discoid lupus erythematosus (DLE; a chronic inflammatory condition of the skin) or systemic lupus erythematosus (SLE; a chronic inflammatory condition of the body), Sjogren's syndrome (an immune system disorder characterized by dry eyes and mouth), and rheumatoid arthritis in patients whose symptoms do not improve with other treatment. In its autoimmune capacity, HCQ is believed to work by decreasing the activity of the immune system. It is also in consideration as a treatment for endometriosis, an often painful disorder in which tissue that is similar to the endometrium, grows outside of the uterus. Endometriosis most commonly involves the ovaries, fallopian tubes and the tissue lining the pelvis.

While HCQ is considered a safe and mostly well-tolerated medication, some patients experience side effects such as headache, dizziness, rash and/or gastrointestinal upset including loss of appetite, nausea, diarrhea, stomach pain, and vomiting. Some patients can experience more severe side effects, such as difficulty reading or seeing (words, letters, or parts of objects missing), sensitivity to light, blurred vision, changes in vision, light flashes or streaks, difficulty hearing, ringing in the ears, muscle weakness, unusual bleeding or bruising, bleaching or loss of hair, mood or mental changes, irregular heartbeat, QT prolongation, drowsiness, convulsions, and/or neurological conditions including decreased consciousness, loss of consciousness, or suicidal ideation. The most serious side effect of HCQ is retinopathy, which can develop after long-term use and cumulative doses of 0.5 kg-1 kg.

Most often, HCQ is used at the FDA approved dosage form containing 200 mg HCQ sulfate per tablet.

### SUMMARY

The present disclosure provides HCQ-containing pharmaceutical compositions for use in the treatment of various diseases including, for example, autoimmune diseases, retinopathy and endometriosis. The disclosed compositions provide for low-dose HCQ treatment of each disease state and/or for adjustable dosing that can be tailored to an individual patient. In various aspects, dosing provided by the disclosed pharmaceutical compositions reduces the chance for a patient to develop side effects from long-term administration of HCQ, while also treating the disease in question. For example, pharmaceutical compositions provided by the present disclosure combine HCQ, or a pharmaceutically acceptable salt thereof, with a coating on the dosage form (e.g., an enteric coating); in an extended release formulation that provides for decreased contact with the GI mucosa; in an oil-based matrix; with sodium bicarbonate or other pH neutralizing agent; and/or in combinations of any of the foregoing.

In addition, pharmaceutical compositions provided by the present disclosure may be formulated as combination dosage forms, containing an active ingredient that is known to treat and/or reduce one or more symptoms of a disease such as retinopathy or endometriosis, in addition to HCQ. The present disclosure also provides for pharmaceutical compositions that are formulated for co-administration with a second dosage form containing an active ingredient that is known to treat and/or reduce one or more symptoms of a disease such as retinopathy or endometriosis. Such dosage forms allow for treatment of such diseases with lower doses of HCQ, reducing the patient's risk of developing HCQ-associated side effects from long-term exposure.

In a first aspect, the present disclosure provides a method of treating endometriosis, comprising: administering a pharmaceutical composition to a subject in need thereof, the pharmaceutical composition comprising: hydroxychloroquine, or a pharmaceutically acceptable salt thereof, in an amount based on the body weight of the subject; and optionally, a pH neutralizing agent; wherein the amount of hydroxychloroquine administered reduces the risk of hydroxychloroquine toxicity in the subject. In embodiments of this first aspect, the subject is very thin and the amount of hydroxychloroquine administered is based on the subject's actual body weight; or the subject is obese or very short and the amount of hydroxychloroquine administered is based on a weight that is less than the subject's actual body weight. In other embodiments of this first aspect, the hydroxychloroquine, or a pharmaceutically acceptable salt thereof, is hydroxychloroquine sulfate; and the amount of hydroxychloroquine sulfate administered is selected from <6.5 mg/kg of the subject's ideal body weight, and <5 mg/kg of the subject's actual body weight. Alternatively, the amount of hydroxychloroquine sulfate administered is <5 mg/kg of the subject's actual body weight. In further embodiments of this first aspect, either: the pharmaceutical composition further comprises vitamin D, zinc, magnesium, melatonin, omega-3 oils, propolis, quercetin, curcumin, N-acetylcysteine, probiotics, resveratrol, alpha lipoic acid, vitamin C, vitamin E, selenium, dichloroacetate, pentoxifylline, epigallocatechin-3-gallate, or combinations thereof; or the pharmaceutical composition is co-administered with a second dosage form comprising vitamin D, zinc, magnesium, melatonin, omega-3 oils, propolis, quercetin, curcumin, N-acetylcysteine, probiotics, resveratrol, alpha lipoic acid, vitamin C, vitamin E, selenium, dichloroacetate, pentoxifylline, epigallocatechin-3-gallate, or combinations thereof.

In a second aspect, the present disclosure provides a method of treating retinopathy, comprising: administering a pharmaceutical composition to a subject in need thereof, the pharmaceutical composition comprising: hydroxychloroquine, or a pharmaceutically acceptable salt thereof, in an amount based on the body weight of the subject; and optionally, a pH neutralizing agent; wherein the amount of hydroxychloroquine administered reduces the risk of hydroxychloroquine toxicity in the subject. In embodiments of this second aspect, the subject is very thin and the amount of hydroxychloroquine administered is based on the subject's actual body weight; or the subject is obese or very short and the amount of hydroxychloroquine administered is based on a weight that is less than the subject's actual body weight. In other embodiments of this second aspect, the hydroxychloroquine, or a pharmaceutically acceptable salt thereof, is hydroxychloroquine sulfate; and the amount of hydroxychloroquine sulfate administered is selected from <6.5 mg/kg of the subject's ideal body weight, and <5 mg/kg of the subject's actual body weight. Alternatively, the amount of hydroxychloroquine sulfate administered is <5 mg/kg of the subject's actual body weight. In further embodiments of this second aspect, either: the pharmaceutical composition further comprises a compound protective of retinal health selected from antioxidants, carotenoids, and combinations thereof, wherein the antioxidants are selected from vitamin C, Vitamin E, acetyl cysteine, and combinations thereof and/or the carotenoids are selected from lutein, zeaxanthin, β-cryptoxanthin, astaxantin, zinc, and combinations thereof; or the pharmaceutical composition is co-administered with a second dosage form comprising: an antioxidant selected from vitamin C, Vitamin E, acetyl cysteine, and combinations thereof; a carotenoid selected from lutein, zeaxanthin, β-cryptoxanthin, astaxantin, zinc, and combinations thereof; or both of the antioxidant and the carotenoid.

In a third aspect, the present disclosure provides a pharmaceutical composition, comprising:

hydroxychloroquine, or a pharmaceutically acceptable salt thereof, in an amount selected from 130 mg and 160 mg; and optionally, a pH neutralizing agent. In some embodiments of this third aspect, the hydroxychloroquine, or a pharmaceutically acceptable salt thereof, is hydroxychloroquine sulfate. In other embodiments of this third aspect, the hydroxychloroquine, or a pharmaceutically acceptable salt thereof, is hydroxychloroquine sulfate; the amount of hydroxychloroquine sulfate is 130 mg; and the pharmaceutical composition comprises about 100.75 mg hydroxychloroquine free base equivalents. In still other embodiments of this third aspect, the hydroxychloroquine, or a pharmaceutically acceptable salt thereof, is hydroxychloroquine sulfate; the amount of hydroxychloroquine sulfate is 160 mg; and the pharmaceutical composition comprises about 124 mg hydroxychloroquine free base equivalents.

In a fourth aspect, the present disclosure provides a packaged pharmaceutical product, comprising: a. a first pharmaceutical composition selected from the group consisting of: i. a pharmaceutical composition comprising hydroxychloroquine sulfate in an amount selected from 130 mg and 160 mg; ii. an enterically coated tablet comprising hydroxychloroquine sulfate in an amount selected from 130 mg and 160 mg; iii. a tablet comprising 100.75 mg or 124 mg hydroxychloroquine free base equivalents in an oil-based matrix; iv. a pharmaceutical composition comprising hydroxychloroquine sulfate in an amount selected from 130 mg and 160 mg, and sodium bicarbonate; and v. a controlled release formulation pharmaceutical composition comprising hydroxychloroquine sulfate in an amount selected from 130 mg and 160 mg; and b. a second pharmaceutical composition that is either protective of retinal health, or beneficial for patients suffering from endometriosis.

### DETAILED DESCRIPTION

### Definitions

“Hydroxychloroquine” (2-[{4-[(7-chloroquinolin-4-yl)amino]pentyl}(ethyl)amino]ethanol) and/or “HCQ” is a compound of the following formula:

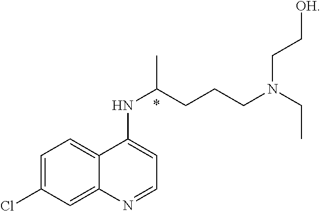 [SMILES: CCN(CCO)CCCC(C)Nc1ccnc2cc(Cl)ccc12]

The asterisk in the above structure denotes a chiral center, indicating that HCQ can exist in two enantiomeric forms, (R)-(−)-2-[{4-[(7-chloroquinolin-4-yl)amino]pentyl}(ethyl)amino]ethanol) and(S)-(+)-2-[{4-[(7-chloroquinolin-4-yl)amino]pentyl} (ethyDamino]ethanol). Accordingly, as used herein, the terms “hydroxychloroquine” and/or “HCQ” encompass both possible enantiomers, including racemic mixtures and enantiomerically pure or enriched forms. Enantiomeric mixtures can be resolved into their component enantiomers using separation techniques or chiral synthesis techniques well known to the skilled artisan.

As used herein, the terms “patient” and “subject” are interchangeable and generally refer to an animal or a human to which a formulation and/or dosage form disclosed herein is administered or is to be administered.

As used herein, the term “pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, lauric acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, pamoic acid, palmitic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like. Unless stated otherwise herein, or unless the context clearly requires otherwise, reference herein to HCQ encompasses HCQ and pharmaceutically acceptable salts thereof.

Hydroxychloroquine, originally developed as an antimalarial agent, has beneficial effects on symptoms in autoimmune disorders, retinopathy and endometriosis. A number of rare but serious adverse reactions can occur with its use, however, and are usually associated with long-term therapy. Common side effects include headache, dizziness, loss of appetite, nausea, diarrhea, stomach pain, vomiting, rash, bleaching of the hair and/or hair loss, and tinnitus, among others. Administration of lower doses of HCQ can typically delay, or even prevent, the onset of side effects in a patient. Many patients take too much HCQ in a single dose, because of the lack of adequate, or varied dose, treatment options available. However, reduced dosing is often associated with a lack of efficacy and so may not be optimal for all patients. The present disclosure solves this problem by providing low dose formulations and/or compositions that allow for tailored, patient-specific dosing. Additionally, in those embodiments in which HCQ is co-administered with an additional active ingredient, HCQ dosing can be reduced significantly without a concomitant reduction in therapeutic efficacy.

### Pharmaceutical Compositions

The present disclosure provides pharmaceutical compositions comprising hydroxychloroquine (HCQ), or pharmaceutically acceptable salts thereof, that demonstrate improved tolerability in patients, for example by reducing or eliminating the occurrence of one or more side effects, or reducing the severity of one or more side effects. In various aspects, the disclosed pharmaceutical compositions comprise HCQ as an active ingredient in admixture with one or more pharmaceutically-acceptable carriers and, optionally, with one or more other compounds, drugs or other materials. Each carrier must be “acceptable” in the sense of being compatible with the other ingredients of the formulation and not injurious to the patient. Pharmaceutically-acceptable carriers are well known in the art. Regardless of the route of administration selected, the compounds of the present disclosure are formulated into pharmaceutically-acceptable dosage forms by conventional methods known to those of skill in the art (see, e.g., Remington's Pharmaceutical Sciences, Philadelphia College of Pharmacy and Science, 17th Edition, 1985).

The disclosed pharmaceutical compositions may be manufactured by means of conventional mixing, dissolving, granulating, dragee-making, levigating, emulsifying, encapsulating, entrapping or lyophilizing processes. Pharmaceutical compositions may be formulated in one or more conventional manners, using one or more physiologically acceptable carriers, diluents, excipients or auxiliaries, which facilitate processing of compounds of the invention into preparations which can be used pharmaceutically.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the dosage form is a capsule; in various embodiments, the dosage form is an orally administrable tablet. Other examples of suitable pharmaceutical vehicles have been described in the art (see, e.g., Remington's Pharmaceutical Sciences, supra). Preferred compositions are formulated for oral delivery.

Compositions for oral delivery may be in the form of tablets, lozenges, aqueous or oily suspensions, granules, powders, emulsions, capsules, syrups, elixirs, sachets, pills, granules, as a solution or a suspension in an aqueous or non-aqueous liquid, an oil-in-water or water-in-oil liquid emulsion, as an elixir or syrup, as pastilles (using an inert base, such as gelatin and glycerin, or sucrose and acacia), or other suitable forms each containing a predetermined amount of HCQ as an active ingredient. Orally administered compositions may contain one or more optional agents, for example, sweetening agents such as fructose, aspartame or saccharin, flavoring agents such as peppermint, oil of wintergreen, or cherry coloring agents and preserving agents, to provide a pharmaceutically palatable preparation.

In solid dosage forms for oral administration (capsules, tablets, pills, dragees, powders, granules and the like), HCQ is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monosterate; (8) absorbents, such as kaolin and bentonite clay; (9) lubricants, such as talc, calcium stearate, magnesium stearate, solid polyethylene glycols, sodium lauryl sulfate, and mixtures thereof; and/or (10) coloring agents. In the case of capsules, tablets and pills, the pharmaceutical compositions may also comprise buffering agents. Solid compositions of a similar type may be employed as fillers in soft and hard-filled gelatin capsules using such excipients as lactose or milk sugars, as well as high molecular weight polyethylene glycols and the like.

In certain embodiments, a dosage form is a tablet, which can be of any shape suitable for oral administration of HCQ, such as spheroidal, cube-shaped oval, or ellipsoidal. A tablet can comprise a unit dose of HCQ, or can comprise less than a unit dose of HCQ in the case of a mini-tablet; in some embodiments, mini-tablets can be filled into capsules to provide a unit dose. In certain embodiments, a tablet can be a multilayer tablet in which different layers contain different particle populations and/or different excipients that affect the release properties of HCQ from each tablet layer. Examples of tablets include disintegrating tablets, fast dissolving tablets, effervescent tablets, fast melt tablets, chewable tablets, crushable tablets, and mini-tablets. Generally, tablet dosage forms can be compressed to a hardness of at least about 15 kiloponds (kp) (equal to 147.1 Newtons). Dosage forms can be prepared in a manner known in the art and can further include pharmaceutically acceptable excipients, as appropriate.

A tablet may be made by compression or molding optionally with one or more accessory ingredients. Compressed tablets may be prepared using binder (for example, gelatin or hydroxypropyl methylcellulose), lubricant, inert diluent, preservative, disintegrant (for example, sodium starch glycolate or cross-linked sodium carboxymethyl cellulose), surface- active or dispersing agent. Molded tablets may be made by molding in a suitable machine a mixture of the powdered compound moistened with an inert liquid diluent. These compositions may also optionally contain opacifying agents and may be of a composition that they release the active ingredient only, or preferentially, in a certain portion of the gastrointestinal tract, optionally, in a delayed manner. Examples of embedding compositions which can be used include polymeric substances and waxes. The active ingredient can also be microencapsulated.

Moreover, where in tablet and/or other solid dosage forms (dragees, capsules, pills, granules, etc.), the compositions may be coated to delay disintegration and absorption in the gastrointestinal tract, thereby providing a sustained action over an extended period of time. In some embodiments, tablets may be prepared with coatings and/or shells, such as enteric coatings and other coatings. They may also be formulated so as to provide slow or controlled release of the active ingredient therein using, for example, hydroxypropyl methylcellulose in varying proportions to provide the desired release profile, other polymer matrices, liposomes and/or microspheres. Selectively permeable membranes surrounding an osmotically active driving compound are also suitable for orally administered compounds and compositions of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

In some embodiments, the present disclosure provides a pharmaceutical composition comprising an enteric coating or other controlled release formulation to reduce direct exposure of the gastric mucosa of a patient to HCQ upon ingestion by circumventing high local HCQ concentrations in the gastrointestinal tract. Enteric coatings are known in the field and are based on polymers, such as cellulose acetate phthalate, hydroxypropyl methylcellulose phthalate, polyvinyl acetate phthalate, acrylic polymers or polyhydroxy acids, such as polyglycolic or polylactic acids. Such coatings are designed to prevent or reduce the disintegration of the tablets and dissolution of the active pharmaceutical ingredient, in this case HCQ, during the transit through the stomach. Such coatings are stable at the acidic pH present within the stomach, but soluble at pH conditions outside of the stomach such as at pH >6.

Compositions for administration via other routes may also be contemplated. For buccal administration, the compositions may take the form of tablets, lozenges, etc. formulated in any conventional manner. Liquid drug formulations suitable for use with nebulizers, liquid spray devices, and electrohydrodynamic aerosol devices will typically include HCQ with a pharmaceutically acceptable vehicle such as a liquid (alcohol, water, polyethylene glycol or a perfluorocarbon). Optionally, another material may be added to alter the aerosol properties of the solution or suspension of HCQ. The disclosed pharmaceutical compositions may also be formulated in rectal or vaginal compositions such as suppositories or retention enemas, e.g., containing conventional suppository bases such as cocoa butter or other glycerides. In addition to the formulations described above, a pharmaceutical composition may also be formulated as a depot preparation. Such long-acting formulations may be administered by implantation (for example subcutaneously) or by intramuscular injection. Thus, for example, pharmaceutical compositions may be formulated with suitable polymeric or hydrophobic materials (for example as an emulsion in an acceptable oil) or ion exchange resins, or as sparingly soluble derivatives, for example, as a sparingly soluble salt.

When a compound of the invention is acidic, it may be included in any of the above-described formulations as the free acid, a pharmaceutically acceptable salt, a solvate or hydrate. Pharmaceutically acceptable salts substantially retain the activity of the free acid, may be prepared by reaction with bases and tend to be more soluble in aqueous and other protic solvents than the corresponding free acid form. Thus, in some embodiments, pharmaceutical compositions comprise HCQ in its free base form and/or wherein the composition comprises an oil-based matrix. When HCQ is in the form of a salt, e.g., as HCQ sulfate, the HCQ is more water soluble and resembles a cationic surfactant than when it is in free base form. Solubilized HCQ is believed to irritate the gastric mucosa, contributing to the GI complaints known for this drug. Thus, formulations of HCQ in free base form and in an oil-based matrix provide improved tolerability when administered to patients.

In further embodiments, pharmaceutical compositions comprise HCQ in combination with sodium bicarbonate or other pH neutralizing agent. Such formulations temporarily neutralize the stomach acid and allow the active ingredient to pass through the stomach, with reduced stomach irritation.

Additional embodiments address the risk of retinal damage by exposure to HCQ by formulating HCQ in a pharmaceutical composition in combination with one or more active ingredients that are protective of retinal health. This type of combination dosage form will potentiate efficacy and/or support to further reduce the dose of HCQ needed to treat retinopathy. Suitable active ingredients that are protective of retinal health include antioxidants (such as vitamin C, vitamin E, acetyl cysteine), carotenoids (such as lutein, zeaxanthin, β-cryptoxanthin, astaxantin), zinc, and combinations thereof. A further embodiment includes the co-administration of HCQ with one or more active ingredients that are protective of retinal health, whether in a single formulation or by administration of HCQ and such an active ingredient independently but at or about at the same time or in a coordinated dosing schedule. Suitable active ingredients in this embodiment include, without limitation, ranibizumab, aflibercept, bevacizumab, and the like.

Another embodiment is a packaged pharmaceutical product that includes a pharmaceutical composition that is protective of retinal health and one or more of a pharmaceutical composition comprising HCQ, pharmaceutical composition comprising HCQ with an enteric coating, a pharmaceutical composition comprising HCQ in free base form in an oil-based matrix, a pharmaceutical composition comprising HCQ and a neutralizing agent such as sodium bicarbonate, a pharmaceutical composition comprising HCQ in a controlled release formulation, and/or combinations thereof.

Additional embodiments address the treatment of endometriosis by formulating HCQ in a pharmaceutical composition in combination with one or more active ingredients that are beneficial for patients suffering from endometriosis. This type of combination dosage form will potentiate efficacy and/or support to further reduce the dose of HCQ needed to treat endometriosis. Suitable active ingredients that are beneficial for patients suffering from endometriosis include vitamin D, zinc, magnesium, melatonin, omega-3 oils, propolis, quercetin, curcumin, N-acetylcysteine, probiotics, resveratrol, alpha lipoic acid, vitamin C, vitamin E, selenium, dichloroacetate, pentoxifylline, epigallocatechin-3-gallate, and combinations thereof. A further embodiment includes the co-administration of HCQ with one or more active ingredients that are that are beneficial for patients suffering from endometriosis, whether in a single formulation or by administration of HCQ and such an active ingredient independently but at or about at the same time or in a coordinated dosing schedule. Suitable active ingredients in this embodiment include, without limitation, vitamin D, zinc, magnesium, melatonin, omega-3 oils, propolis, quercetin, curcumin, N-acetylcysteine, probiotics, resveratrol, alpha lipoic acid, vitamin C, vitamin E, selenium, dichloroacetate, pentoxifylline, epigallocatechin-3-gallate, and combinations thereof.

### Dosing

The disclosed HCQ pharmaceutical compositions will generally be administered in an amount effective to achieve the intended purpose. For use to treat or prevent diseases or disorders such as lupus, Sjogren's syndrome, rheumatoid arthritis, retinopathy, endometriosis, or other, related disorders HCQ is administered or applied in a therapeutically effective amount.

The amount of a compound of HCQ that will be effective in the treatment of a particular disorder or condition disclosed herein will depend on the nature of the disorder or condition. The amount of HCQ administered to an individual patient will, of course, be dependent on the subject being treated, the weight of the subject, the severity of the affliction, the manner of administration, and the judgment of the prescribing physician, among other factors.

For example, the dosage may be delivered in a pharmaceutical composition by a single administration, by multiple administrations, and/or controlled release. In a preferred embodiment, HCQ is administered to a patient orally, for example in a tablet dosage form. Preferably, in this embodiment, HCQ is administered to a patient once per day, more than once per day, repeated intermittently, and/or alone or in combination with other drugs, and may continue as long as required for effective treatment of the disease state or disorder.

In various aspect, dosing of patients with HCQ is individualized in order to minimize the potential side effects of HCQ administration, as well as to properly treat the chronic nature of the conditions it is administered to treat. Long term treatment of such conditions can result in significant lifetime accumulated exposure to the drug. Moreover, very thin patients are at risk of receiving higher than recommended dosing if they are dosed based on ideal body weight (IBW) which may not be reflective of their actual body weight. Contrarily, obese or very short patients run the risk of renal toxicity if they are dosed based on actual body weight (ABW). The traditional dosage recommendation for administration of hydroxychloroquine sulfate is <6.5 mg/kg of Ideal Body Weight (IBW), but the current American Academy of Ophthalmology (AAO) recommendation, taking into account risk of retinal damage from hydroxychloroquine sulfate, is <5 mg/kg of Actual Body Weight (ABW). Administration of the appropriate dosage to achieve an adequate pharmacologic response while maintaining an acceptable risk of side effects is complicated and should account for an individual patient's body type, the condition being treated, and the relevant risk of side effects. To date, HCQ is only available in 200 mg tablets of hydroxychloroquine sulfate, which is problematic. Pediatric patients with ABW <31 kg would exceed the recommended dose (<5 mg/kg of ABW) if they took a single 200 mg tablet each day. Thus, the present disclosure provides HCQ pharmaceutical dosage forms that allow for more flexible, individualized dosing.

Accordingly, an embodiment of the disclosure is a pharmaceutical dosage form comprising HCQ wherein the dosage form is a scored tablet. Such a dosage form allows for the ready fractionation of the tablet resulting in two or more subparts of the tablet, each subpart containing a smaller dose than the complete tablet so that fractional doses can be administered. For example, tablets can be scored into two halves (each half containing half of the dose of the tablet) or into four quarters (each quarter containing ¼ of the dose of the tablet). Such dosage forms of scored tablets can have a hydroxychloroquine sulfate content selected from 130 mg to 200 mg per tablet, 130 mg to 375 mg per tablet, or 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, or any other whole number of mg between 130 and 375 or any range of whole numbers between 130 and 375 per tablet. In alternative embodiments, the dosage form of scored tablets have a hydroxychloroquine sulfate content selected from about 130 mg hydroxychloroquine sulfate, about 160 mg hydroxychloroquine sulfate, about 180 mg hydroxychloroquine sulfate, about 200 mg hydroxychloroquine sulfate, or about 375 mg hydroxychloroquine sulfate. In various aspects, dosing per patient in the pharmaceutical compositions provided by the present disclosure follows AAO recommended dosing per patient, which does not exceed 5 mg/Kg ABW per day.

In alternative embodiments, dosage forms of the disclosure can be described in terms of hydroxychloroquine equivalents to hydroxychloroquine sulfate amounts. The equivalent amount of free base hydroxychloroquine to the amount of hydroxychloroquine in a 200 mg dose of hydroxychloroquine sulfate is 155 mg of hydroxychloroquine. Other salts of hydroxychloroquine (non-sulfate salts) having, for example, 155 mg of hydroxychloroquine, will have a total weight different from 155 mg and 200 mg depending on the molecular weight of the cation. Thus, other dosage forms of scored tablets can have a hydroxychloroquine content selected from 100.75 mg to 155 mg, 100.75 mg to 290.6 mg, or any whole number of mg between 100.75 and 290.6 or any range of whole numbers between 100.75 and 290.6. In alternative embodiments, the dosage form of scored tablets can have about 100.75 mg hydroxychloroquine, about 124 mg hydroxychloroquine, about 139.4 mg hydroxychloroquine, about 155 mg hydroxychloroquine, or about 290.6 mg hydroxychloroquine, all as free base equivalents.

Table 1 illustrates the HCQ content of subparts of tablets having various starting HCQ contents. It should be recognized that all dosages referred to herein of hydroxychloroquine sulfate are intended to also convey the corresponding amount of hydroxychloroquine equivalents which can be determined by multiplying the hydroxychloroquine amount by the fraction of 155/200 or 0.775.

TABLE 1

| Total | Hydroxychloroquine | Hydroxychloroquine |
|---|---|---|
| Hydroxychloroquine | Sulfate | Sulfate |
| Sulfate | (hydroxychloroquine) | (hydroxychloroquine) |
| (hydroxychloroquine) | Content/ | Content/ |
| Content (mg) | half (mg) | quarter (mg) |
| 375 (290.6) | 187.5 (145.3) | 93.75 (72.7) |
| 200 (155) | 100 (77.5) | 50 (38.8) |
| 180 (139.4) | 90 (69.7) | 45 (34.9) |
| 160 (124) | 80 (62) | 40 (31) |
| 130 (100.75) | 65 (50.37) | 32.5 (25.2) |

For example, a hydroxychloroquine sulfate tablet of 200 mg (breakable into two 100 mg halves or 50 mg quarters) provides 5 mg/kg for a 40 kg (88 lb) patient; a tablet of 180 mg (breakable into two 90 mg halves or 45 mg quarters) provides about 5 mg/kg for a 35 kg (79 lb) patient; a tablet of 160 mg (breakable into two 80 mg halves or 40 mg quarters) provides about 5 mg/kg for a 31 kg (70 lb) patient; and a tablet of 130 mg (breakable into two 65 mg halves or 32.55 mg quarters) provides about 5 mg/kg for a 26 kg (57 lb) patient. Further, for example, a patient that should be dosed at 5 mg/kg that weights 50 kg (110 lb) can take a whole 200 mg tablet plus one quarter of a 200 mg tablet for a total dose of 250 mg. In this manner, more precise dosing of patients can be achieved with dosage forms of the present disclosure.

The above analysis would also apply to disclosed dosage forms that include HCQ in combination with another active ingredient. The total dose of the other active ingredient could similarly be halved and/or quartered when supplied as part of the scored tablet dosage forms provided by the present disclosure. In that regard, various HCQ-containing formulations are disclosed herein, such as pharmaceutical compositions comprising HCQ and having an enteric coating. In addition, various dosage forms having low doses of HCQ are disclosed. It should be appreciated that the present disclosure contemplates the use of any of the various disclosed dosage forms in combination with any of the disclosed pharmaceutical formulations. For example, a pharmaceutical composition comprising HCQ and sodium bicarbonate can have HCQ present in any concentration disclosed herein, such as a content of between 130 mg and 200 mg of hydroxychloroquine sulfate.

### Indications, Methods of Use

The present disclosure provides methods of treating a condition in a patient by administration of one or more hydroxychloroquine-containing pharmaceutical compositions disclosed herein. Such methods comprise, inter alia, administering hydroxychloroquine sulfate at a dosage of less than 6.5 mg hydroxychloroquine sulfate/kg/day, less than 6.0 mg hydroxychloroquine sulfate/kg/day, less than 5.5 mg hydroxychloroquine sulfate/kg/day, less than 5.0 mg hydroxychloroquine sulfate/kg/day, less than 4.5 mg hydroxychloroquine sulfate/kg/day, less than 4.0 mg hydroxychloroquine sulfate/kg/day, less than 3.5 mg hydroxychloroquine sulfate/kg/day, less than 3.0 mg hydroxychloroquine sulfate/kg/day, less than 2.5 mg hydroxychloroquine sulfate/kg/day, or less than 2.0 mg hydroxychloroquine sulfate/kg/day. Alternatively, such embodiments comprise administering hydroxychloroquine at a dosage of less than 5.0 mg hydroxychloroquine/kg/day, less than 4.7 mg hydroxychloroquine/kg/day, less than 4.3 mg hydroxychloroquine/kg/day, less than 3.9 mg hydroxychloroquine/kg/day, less than 3.5 mg hydroxychloroquine/kg/day, less than 3.1 mg hydroxychloroquine/kg/day, less than 2.7 mg hydroxychloroquine/kg/day, less than 2.3 mg hydroxychloroquine/kg/day, less than 1.9 mg hydroxychloroquine/kg/day, or less than 1.6 mg hydroxychloroquine/kg/day. Suitable conditions for treatment of a patient by administration of hydroxychloroquine include retinopathy, autoimmune disorders and endometriosis. Autoimmune disorders suitable for treatment by the disclosed pharmaceutical compositions include lupus, Sjogren's syndrome, and rheumatoid arthritis.

### Lupus

Lupus is an autoimmune disease characterized by often severe inflammation in many different body systems, including joints, skin, kidneys, blood cells, brain, heart and lungs. Symptoms vary from patient to patient and may come on suddenly or develop slowly, may be mild or severe, and/or may be temporary or permanent. The vast majority of lupus patients experience a mild form of the disease characterized by flares when signs and symptoms get worse for a short while, then improve or even disappear completely for a time. The symptoms of lupus depend on which body systems are affected by the disease. The most common signs and symptoms include: fatigue, fever, joint pain, stiffness and swelling, butterfly-shaped rash on the face that covers the cheeks and bridge of the nose or rashes elsewhere on the body, skin lesions that appear or worsen with sun exposure, fingers and toes that turn white or blue when exposed to cold or during stressful periods, shortness of breath, chest pain, dry eyes, headaches, confusion and memory loss.

Pharmaceutical compositions provided by the present disclosure are useful in the low-dose HCQ treatment of lupus. In some embodiments, the present disclosure provides methods for the prophylactic use of HCQ-containing pharmaceutical compositions of the disclosure to reduce the likelihood of developing, or reduce the severity of one or more symptoms of lupus. In that regard, the present disclosure provides methods of treating lupus in a patient by administration of one or more hydroxychloroquine-containing pharmaceutical compositions disclosed herein. Such methods comprise, inter alia, administering hydroxychloroquine sulfate at a dosage of less than 6.5 mg hydroxychloroquine sulfate/kg/day, less than 6.0 mg hydroxychloroquine sulfate/kg/day, less than 5.5 mg hydroxychloroquine sulfate/kg/day, less than 5.0 mg hydroxychloroquine sulfate/kg/day, less than 4.5 mg hydroxychloroquine sulfate/kg/day, less than 4.0 mg hydroxychloroquine sulfate/kg/day, less than 3.5 mg hydroxychloroquine sulfate/kg/day, less than 3.0 mg hydroxychloroquine sulfate/kg/day, less than 2.5 mg hydroxychloroquine sulfate/kg/day, or less than 2.0 mg hydroxychloroquine sulfate/kg/day. Alternatively, such embodiments comprise administering hydroxychloroquine at a dosage of less than 5.0 mg hydroxychloroquine/kg/day, less than 4.7 mg hydroxychloroquine/kg/day, less than 4.3 mg hydroxychloroquine/kg/day, less than 3.9 mg hydroxychloroquine/kg/day, less than 3.5 mg hydroxychloroquine/kg/day, less than 3.1 mg hydroxychloroquine/kg/day, less than 2.7 mg hydroxychloroquine/kg/day, less than 2.3 mg hydroxychloroquine/kg/day, less than 1.9 mg hydroxychloroquine/kg/day, or less than 1.6 mg hydroxychloroquine/kg/day.

### Sjogren's Syndrome

Sjogren's syndrome is an autoimmune disorder characterized by two most common symptoms, dry eyes and a dry mouth. The condition often accompanies other autoimmune disorders, such as rheumatoid arthritis and lupus. In Sjogren's syndrome, the mucous membranes and moisture-secreting glands of a patient's eyes and mouth are usually affected first, resulting in decreased tears and saliva. Although one can develop Sjogren's syndrome at any age, most people are older than 40 at the time of diagnosis. The condition is much more common in women and treatment focuses on relieving symptoms.

Pharmaceutical compositions provided by the present disclosure are useful in the low-dose HCQ treatment of Sjogren's syndrome. In some embodiments, the present disclosure provides methods for the prophylactic use of HCQ-containing pharmaceutical compositions of the disclosure to reduce the likelihood of developing, or reduce the severity of one or more symptoms of Sjogren's syndrome. In that regard, the present disclosure provides methods of treating Sjogren's syndrome in a patient by administration of one or more hydroxychloroquine-containing pharmaceutical compositions disclosed herein. Such methods comprise, inter alia, administering hydroxychloroquine sulfate at a dosage of less than 6.5 mg hydroxychloroquine sulfate/kg/day, less than 6.0 mg hydroxychloroquine sulfate/kg/day, less than 5.5 mg hydroxychloroquine sulfate/kg/day, less than 5.0 mg hydroxychloroquine sulfate/kg/day, less than 4.5 mg hydroxychloroquine sulfate/kg/day, less than 4.0 mg hydroxychloroquine sulfate/kg/day, less than 3.5 mg hydroxychloroquine sulfate/kg/day, less than 3.0 mg hydroxychloroquine sulfate/kg/day, less than 2.5 mg hydroxychloroquine sulfate/kg/day, or less than 2.0 mg hydroxychloroquine sulfate/kg/day. Alternatively, such embodiments comprise administering hydroxychloroquine at a dosage of less than 5.0 mg hydroxychloroquine/kg/day, less than 4.7 mg hydroxychloroquine/kg/day, less than 4.3 mg hydroxychloroquine/kg/day, less than 3.9 mg hydroxychloroquine/kg/day, less than 3.5 mg hydroxychloroquine/kg/day, less than 3.1 mg hydroxychloroquine/kg/day, less than 2.7 mg hydroxychloroquine/kg/day, less than 2.3 mg hydroxychloroquine/kg/day, less than 1.9 mg hydroxychloroquine/kg/day, or less than 1.6 mg hydroxychloroquine/kg/day.

### Rheumatoid Arthritis

Rheumatoid arthritis (commonly known as “RA”) is an autoimmune and inflammatory disease, causing inflammation (painful swelling) in the affected parts of the body. RA mainly attacks the joints, usually multiple joints at one time, most often in the hands, wrists, and knees. Typically, an RA-inflicted joint becomes severely inflamed, causing damage to joint tissue. This tissue damage can cause long-lasting or chronic pain, unsteadiness (lack of balance), and deformity (misshapenness). RA can also affect other tissues throughout the body and cause problems in organs such as the lungs, heart, and eyes.

Signs and symptoms of RA include pain or aching in more than one joint, stiffness in more than one joint, tenderness and swelling in more than one joint, the same symptoms on both sides of the body (such as in both hands or both knees), weight loss, fever, fatigue or tiredness, weakness, and/or combinations of any of the foregoing.

Pharmaceutical compositions provided by the present disclosure are useful in the low-dose HCQ treatment of RA. In some embodiments, the present disclosure provides methods for the prophylactic use of HCQ-containing pharmaceutical compositions of the disclosure to reduce the likelihood of developing, or reduce the severity of one or more symptoms of RA. In that regard, the present disclosure provides methods of treating RA in a patient by administration of one or more hydroxychloroquine-containing pharmaceutical compositions disclosed herein. Such methods comprise, inter alia, administering hydroxychloroquine sulfate at a dosage of less than 6.5 mg hydroxychloroquine sulfate/kg/day, less than 6.0 mg hydroxychloroquine sulfate/kg/day, less than 5.5 mg hydroxychloroquine sulfate/kg/day, less than 5.0 mg hydroxychloroquine sulfate/kg/day, less than 4.5 mg hydroxychloroquine sulfate/kg/day, less than 4.0 mg hydroxychloroquine sulfate/kg/day, less than 3.5 mg hydroxychloroquine sulfate/kg/day, less than 3.0 mg hydroxychloroquine sulfate/kg/day, less than 2.5 mg hydroxychloroquine sulfate/kg/day, or less than 2.0 mg hydroxychloroquine sulfate/kg/day. Alternatively, such embodiments comprise administering hydroxychloroquine at a dosage of less than 5.0 mg hydroxychloroquine/kg/day, less than 4.7 mg hydroxychloroquine/kg/day, less than 4.3 mg hydroxychloroquine/kg/day, less than 3.9 mg hydroxychloroquine/kg/day, less than 3.5 mg hydroxychloroquine/kg/day, less than 3.1 mg hydroxychloroquine/kg/day, less than 2.7 mg hydroxychloroquine/kg/day, less than 2.3 mg hydroxychloroquine/kg/day, less than 1.9 mg hydroxychloroquine/kg/day, or less than 1.6 mg hydroxychloroquine/kg/day.

### Retinopathy

Retinopathy is a disease of the retina. There are several types of retinopathy, for example hypertensive or diabetic retinopathy, all of which involve disease of the small retinal blood vessels (see, e.g., J. Torpy MD, et al., JAMA (2007) 298 (8): 944. doi: 10.1001/jama.298.8.944). Signs of retinopathy (see photograph) can be seen when the retina is viewed through the pupil with an ophthalmoscope. Once retinopathy is detected, early treatment is recommended in order to prevent blindness. Signs and symptoms of retinopathy include blurry vision, double vision, “floaters” or spots in vision, eye pain and redness that does not resolve, decreased peripheral vision, or combinations of the foregoing.

Pharmaceutical compositions provided by the present disclosure are useful in the low-dose HCQ treatment of retinopathy, regardless of type. In some embodiments, the present disclosure provides methods for the prophylactic use of HCQ-containing pharmaceutical compositions of the disclosure to reduce the likelihood of developing, or reduce the severity of one or more symptoms of retinopathy. In that regard, the present disclosure provides methods of treating retinopathy in a patient by administration of one or more hydroxychloroquine-containing pharmaceutical compositions disclosed herein. Such methods comprise, inter alia, administering hydroxychloroquine sulfate at a dosage of less than 6.5 mg hydroxychloroquine sulfate/kg/day, less than 6.0 mg hydroxychloroquine sulfate/kg/day, less than 5.5 mg hydroxychloroquine sulfate/kg/day, less than 5.0 mg hydroxychloroquine sulfate/kg/day, less than 4.5 mg hydroxychloroquine sulfate/kg/day, less than 4.0 mg hydroxychloroquine sulfate/kg/day, less than 3.5 mg hydroxychloroquine sulfate/kg/day, less than 3.0 mg hydroxychloroquine sulfate/kg/day, less than 2.5 mg hydroxychloroquine sulfate/kg/day, or less than 2.0 mg hydroxychloroquine sulfate/kg/day. Alternatively, such embodiments comprise administering hydroxychloroquine at a dosage of less than 5.0 mg hydroxychloroquine/kg/day, less than 4.7 mg hydroxychloroquine/kg/day, less than 4.3 mg hydroxychloroquine/kg/day, less than 3.9 mg hydroxychloroquine/kg/day, less than 3.5 mg hydroxychloroquine/kg/day, less than 3.1 mg hydroxychloroquine/kg/day, less than 2.7 mg hydroxychloroquine/kg/day, less than 2.3 mg hydroxychloroquine/kg/day, less than 1.9 mg hydroxychloroquine/kg/day, or less than 1.6 mg hydroxychloroquine/kg/day.

### Endometriosis

Endometriosis is a common gynecological condition affecting an estimated 2 to 10 percent of Americans capable of bearing children. The name of this condition comes from the word “endometrium,” which is the tissue that lines the uterus. Endometriosis is an often painful disorder in which tissue that is similar to the endometrium, grows outside of the uterus. Endometriosis most commonly involves the ovaries, fallopian tubes and the tissue lining the pelvis.

The present disclosure provides methods for the prophylactic use of HCQ-containing compositions and formulations of the disclosure to reduce the likelihood of developing or reduce the severity of patients developing endometriosis. While the condition is limited to patients capable of bearing children, there are known risk factors for people for development of the condition. For example, the following risk factors are known: family history of endometriosis; early age of menarche; short menstrual cycles (<27 days); long duration of menstrual flow (>7 days); heavy bleeding during menses; inverse relationship to parity:

delayed childbearing; defects in the uterus or fallopian tubes; hypoxia and iron deficiency; and/or combinations of any of the foregoing In addition, it is recognized that endometriosis occurs more frequently in patients having certain other conditions such as autoimmune conditions (e.g., lupus).

The prophylactic use of HCQ includes the administration of HCQ-containing compositions and formulations of the disclosure to patients at risk of developing endometriosis, including patients of child-bearing age and additionally, having one or more risk factors for an elevated risk of developing the condition compared to the population at large. Suitable patients for prophylactic treatment include patients having no symptoms of endometriosis, but who display one or more risk factors. In addition, suitable patients include patients having mild symptoms consistent with endometriosis although possibly without a definitive diagnosis of the condition, as well as diagnosed patients having symptoms not warranting pharmaceutical treatment under conventional treatment protocols. Such prophylactic administration of HCQ can be at conventional doses such as 6.5 mg hydroxychloroquine sulfate/kg of body weight or 5.0 mg hydroxychloroquine sulfate/kg of body weight or at lower doses as disclosed herein.

Pharmaceutical compositions provided by the present disclosure are useful in the low-dose HCQ treatment of endometriosis. In some embodiments, the present disclosure provides methods for the prophylactic use of HCQ-containing pharmaceutical compositions of the disclosure to reduce the likelihood of developing, or reduce the severity of one or more symptoms of endometriosis. In that regard, the present disclosure provides methods of treating endometriosis in a patient by administration of one or more hydroxychloroquine-containing pharmaceutical compositions disclosed herein. Such methods comprise, inter alia, administering hydroxychloroquine sulfate at a dosage of less than 6.5 mg hydroxychloroquine sulfate/kg/day, less than 6.0 mg hydroxychloroquine sulfate/kg/day, less than 5.5 mg hydroxychloroquine sulfate/kg/day, less than 5.0 mg hydroxychloroquine sulfate/kg/day, less than 4.5 mg hydroxychloroquine sulfate/kg/day, less than 4.0 mg hydroxychloroquine sulfate/kg/day, less than 3.5 mg hydroxychloroquine sulfate/kg/day, less than 3.0 mg hydroxychloroquine sulfate/kg/day, less than 2.5 mg hydroxychloroquine sulfate/kg/day, or less than 2.0 mg hydroxychloroquine sulfate/kg/day. Alternatively, such embodiments comprise administering hydroxychloroquine at a dosage of less than 5.0 mg hydroxychloroquine/kg/day, less than 4.7 mg hydroxychloroquine/kg/day, less than 4.3 mg hydroxychloroquine/kg/day, less than 3.9 mg hydroxychloroquine/kg/day, less than 3.5 mg hydroxychloroquine/kg/day, less than 3.1 mg hydroxychloroquine/kg/day, less than 2.7 mg hydroxychloroquine/kg/day, less than 2.3 mg hydroxychloroquine/kg/day, less than 1.9 mg hydroxychloroquine/kg/day, or less than 1.6 mg hydroxychloroquine/kg/day.

The disclosure illustratively disclosed herein suitably may be practiced in the absence of any element which is not specifically disclosed herein. It is apparent to those skilled in the art, however, that many changes, variations, modifications, other uses, and applications of the disclosure are possible, and also changes, variations, modifications, other uses, and applications which do not depart from the spirit and scope of the disclosure are deemed to be covered by the disclosure, which is limited only by the claims which follow. The foregoing discussion of the disclosure has been presented for purposes of illustration and description. The foregoing is not intended to limit the disclosure to the form or forms disclosed herein. In the foregoing Detailed Description, for example, various features of the disclosure are grouped together in one or more embodiments for the purpose of streamlining the disclosure. The features of the embodiments of the disclosure may be combined in alternate embodiments other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

Moreover, though the description of the disclosure has included description of one or more embodiments and certain variations and modifications, other variations, combinations, and modifications are within the scope of the disclosure, e.g., as may be within the skill and knowledge of those in the art, after understanding the present disclosure. It is intended to obtain rights which include alternative embodiments to the extent permitted, including alternate, interchangeable, and/or equivalent structures, functions, ranges, or steps to those claimed, whether or not such alternate, interchangeable, and/or equivalent structures, functions, ranges, or steps are disclosed herein, and without intending to publicly dedicate any patentable subject matter.

What is claimed is:

1. A method of treating endometriosis, comprising:
administering a pharmaceutical composition to a subject in need thereof, the pharmaceutical composition comprising:
hydroxychloroquine, or a pharmaceutically acceptable salt thereof, in an amount based on the body weight of the subject; and
optionally, a pH neutralizing agent;
wherein the amount of hydroxychloroquine administered reduces the risk of hydroxychloroquine toxicity in the subject.

2. The method of claim 1, wherein:
the subject is very thin and the amount of hydroxychloroquine administered is based on the subject's actual body weight; or
the subject is obese or very short and the amount of hydroxychloroquine administered is based on a weight that is less than the subject's actual body weight.

3. The method of claim 1 or claim 2, wherein:
the hydroxychloroquine, or a pharmaceutically acceptable salt thereof, is hydroxychloroquine sulfate; and
the amount of hydroxychloroquine sulfate administered is selected from <6.5 mg/kg of the subject's ideal body weight, and <5 mg/kg of the subject's actual body weight.

4. The method of any one of claims 1-3, wherein the amount of hydroxychloroquine sulfate administered is <5 mg/kg of the subject's actual body weight.

5. The method of any one of claims 1-4, wherein the hydroxychloroquine, or a pharmaceutically acceptable salt thereof, is hydroxychloroquine sulfate and the amount of hydroxychloroquine sulfate administered is selected from: less than 6.5 mg hydroxychloroquine sulfate/kg/day, less than 6.0 mg hydroxychloroquine sulfate/kg/day, less than 5.5 mg hydroxychloroquine sulfate/kg/day, less than 5.0 mg hydroxychloroquine sulfate/kg/day, less than 4.5 mg hydroxychloroquine sulfate/kg/day, less than 4.0 mg hydroxychloroquine sulfate/kg/day, less than 3.5 mg hydroxychloroquine sulfate/kg/day, less than 3.0 mg hydroxychloroquine sulfate/kg/day, less than 2.5 mg hydroxychloroquine sulfate/kg/day, and less than 2.0 mg hydroxychloroquine sulfate/kg/day.

6. The method of claim 1 or claim 2, wherein the amount of hydroxychloroquine administered is selected from: less than 5.0 mg hydroxychloroquine/kg/day, less than 4.7 mg hydroxychloroquine/kg/day, less than 4.3 mg hydroxychloroquine/kg/day, less than 3.9 mg hydroxychloroquine/kg/day, less than 3.5 mg hydroxychloroquine/kg/day, less than 3.1 mg hydroxychloroquine/kg/day, less than 2.7 mg hydroxychloroquine/kg/day, less than 2.3 mg hydroxychloroquine/kg/day, less than 1.9 mg hydroxychloroquine/kg/day, and less than 1.6 mg hydroxychloroquine/kg/day. 7 The method of any one of claims 1-6, wherein the pharmaceutical composition is a tablet comprising an enteric coating.

8. The method of any one of claims 1-6, wherein the pharmaceutical composition is a tablet comprising the hydroxychloroquine in an oil-based matrix.

9. The method of claim 7 or claim 8, wherein the tablet is a controlled release formulation.

10. The method of any one of claims 7-9, wherein the tablet is a scored tablet. 11 The method of any one of claims 7-10, wherein the tablet comprises about 100.75 mg-about 155 mg hydroxychloroquine equivalents.

12. The method of any one of claims 7-11, wherein the tablet comprises about 100.75 mg hydroxychloroquine equivalents.

13. The method of any one of claims 7-11, wherein the tablet comprises about 124 mg hydroxychloroquine equivalents.

14. The method of any one of claims 7-11, wherein the tablet comprises about 139.4 mg hydroxychloroquine equivalents.

15. The method of any one of claims 7-11, wherein the tablet comprises about 155 mg hydroxychloroquine equivalents.

16. The method of any one of claims 7-11, wherein the tablet comprises about 290.6 mg hydroxychloroquine equivalents.

17. The method of any one of claims 7-10, wherein the tablet comprises 130 mg hydroxychloroquine sulfate or 160 mg hydroxychloroquine sulfate.

18. The method of any one of claims 7-17, wherein the tablet is scored into halves.

19. The method of any one of claims 7-17, wherein the tablet is scored into quarters.

20. The method of any one of claims 1-19, wherein the pH neutralizing agent is sodium bicarbonate.

21. The method of any one of claims 1-20, wherein the pharmaceutical composition further comprises vitamin D, zinc, magnesium, melatonin, omega-3 oils, propolis, quercetin, curcumin, N-acetylcysteine, probiotics, resveratrol, alpha lipoic acid, vitamin C, vitamin E, selenium, dichloroacetate, pentoxifylline, epigallocatechin-3-gallate, or combinations thereof.

22. The method of any one of claims 1-20, wherein the pharmaceutical composition is co-administered with a second dosage form comprising vitamin D, zinc, magnesium, melatonin, omega-3 oils, propolis, quercetin, curcumin, N-acetylcysteine, probiotics, resveratrol, alpha lipoic acid, vitamin C, vitamin E, selenium, dichloroacetate, pentoxifylline, epigallocatechin-3-gallate, or combinations thereof.

23. A method of treating retinopathy, comprising:
administering a pharmaceutical composition to a subject in need thereof, the pharmaceutical composition comprising:
hydroxychloroquine, or a pharmaceutically acceptable salt thereof, in an amount based on the body weight of the subject; and
optionally, a pH neutralizing agent;
wherein the amount of hydroxychloroquine administered reduces the risk of hydroxychloroquine toxicity in the subject. 24 The method of claim 23, wherein:
the subject is very thin and the amount of hydroxychloroquine administered is based on the subject's actual body weight; or
the subject is obese or very short and the amount of hydroxychloroquine administered is based on a weight that is less than the subject's actual body weight.

25. The method of claim 23 or claim 24, wherein:
the hydroxychloroquine, or a pharmaceutically acceptable salt thereof, is hydroxychloroquine sulfate; and
the amount of hydroxychloroquine sulfate administered is selected from <6.5 mg/kg of the subject's ideal body weight, and <5 mg/kg of the subject's actual body weight.

26. The method of any one of claims 23-25, wherein the amount of hydroxychloroquine sulfate administered is <5 mg/kg of the subject's actual body weight.

27. The method of any one of claims 23-26, wherein the hydroxychloroquine, or a pharmaceutically acceptable salt thereof, is hydroxychloroquine sulfate and the amount of hydroxychloroquine sulfate administered is selected from: less than 6.5 mg hydroxychloroquine sulfate/kg/day, less than 6.0 mg hydroxychloroquine sulfate/kg/day, less than 5.5 mg hydroxychloroquine sulfate/kg/day, less than 5.0 mg hydroxychloroquine sulfate/kg/day, less than 4.5 mg hydroxychloroquine sulfate/kg/day, less than 4.0 mg hydroxychloroquine sulfate/kg/day, less than 3.5 mg hydroxychloroquine sulfate/kg/day, less than 3.0 mg hydroxychloroquine sulfate/kg/day, less than 2.5 mg hydroxychloroquine sulfate/kg/day, and less than 2.0 mg hydroxychloroquine sulfate/kg/day. 28 The method of claim 23 or claim 24, wherein the amount of hydroxychloroquine administered is selected from: less than 5.0 mg hydroxychloroquine/kg/day, less than 4.7 mg hydroxychloroquine/kg/day, less than 4.3 mg hydroxychloroquine/kg/day, less than 3.9 mg hydroxychloroquine/kg/day, less than 3.5 mg hydroxychloroquine/kg/day, less than 3.1 mg hydroxychloroquine/kg/day, less than 2.7 mg hydroxychloroquine/kg/day, less than 2.3 mg hydroxychloroquine/kg/day, less than 1.9 mg hydroxychloroquine/kg/day, and less than 1.6 mg hydroxychloroquine/kg/day.

29. The method of any one of claims 23-28, wherein the pharmaceutical composition is a tablet comprising an enteric coating.

30. The method of any one of claims 23-28, wherein the pharmaceutical composition is a tablet comprising the hydroxychloroquine in an oil-based matrix.

31. The method of claim 29 or claim 30, wherein the tablet is a controlled release formulation.

32. The method of any one of claims 29-31, wherein the tablet is a scored tablet.

33. The method of any one of claims 29-32, wherein the tablet comprises about 100.75 mg-about 155 mg hydroxychloroquine equivalents.

34. The method of any one of claims 29-33, wherein the tablet comprises about 100.75 mg hydroxychloroquine equivalents.

35. The method of any one of claims 29-33, wherein the tablet comprises about 124 mg hydroxychloroquine equivalents.

36. The method of any one of claims 29-33, wherein the tablet comprises about 139.4 mg hydroxychloroquine equivalents.

37. The method of any one of claims 29-33, wherein the tablet comprises about 155 mg hydroxychloroquine equivalents.

38. The method of any one of claims 29-33, wherein the tablet comprises about 290.6 mg hydroxychloroquine equivalents.

39. The method of any one of claims 29-32, wherein the tablet comprises 130 mg hydroxychloroquine sulfate or 160 mg hydroxychloroquine sulfate.

40. The method of any one of claims 29-39, wherein the tablet is scored into halves.

41. The method of any one of claims 29-39, wherein the tablet is scored into quarters.

42. The method of any one of claims 23-41, wherein the PH neutralizing agent is sodium bicarbonate.

43. The method of any one of claims 23-42, wherein the pharmaceutical composition further comprises a compound protective of retinal health selected from antioxidants, carotenoids, and combinations thereof.

44. The method of claim 43, wherein the antioxidants are selected from vitamin C, Vitamin E, acetyl cysteine, and combinations thereof.

45. The method of claim 43 or claim 44, wherein the carotenoids are selected from lutein, zeaxanthin, β-cryptoxanthin, astaxantin, zinc, and combinations thereof.

46. The method of any one of claims 23-42, wherein the pharmaceutical composition is co-administered with a second dosage form comprising:
an antioxidant selected from vitamin C, Vitamin E, acetyl cysteine, and combinations thereof;
a carotenoid selected from lutein, zeaxanthin, β-cryptoxanthin, astaxantin, zinc, and combinations thereof; or
both of the antioxidant and the carotenoid.

47. A pharmaceutical composition, comprising:
hydroxychloroquine, or a pharmaceutically acceptable salt thereof, in an amount selected from 130 mg and 160 mg; and
optionally, a pH neutralizing agent.

48. The pharmaceutical composition of claim 47, wherein the hydroxychloroquine, or a pharmaceutically acceptable salt thereof, is hydroxychloroquine sulfate.

49. The pharmaceutical composition of claim 47 or claim 48, wherein the pharmaceutical composition is a tablet comprising an enteric coating.

50. The pharmaceutical composition of claim 47 or claim 48, wherein the pharmaceutical composition is a tablet comprising the hydroxychloroquine in an oil-based matrix.

51. The pharmaceutical composition of any one of claims 47-50, wherein the tablet is a controlled release formulation.

52. The pharmaceutical composition of any one of claims 47-51, wherein the tablet is a scored tablet.

53. The pharmaceutical composition of claim 52, wherein the tablet is scored into halves.

54. The pharmaceutical composition of claim 42, wherein the tablet is scored into quarters.

55. The pharmaceutical composition of any one of claims 47-54, wherein:
the hydroxychloroquine, or a pharmaceutically acceptable salt thereof, is hydroxychloroquine sulfate;
the amount of hydroxychloroquine sulfate is 130 mg; and
the pharmaceutical composition comprises about 100.75 mg hydroxychloroquine free base equivalents.

56. The pharmaceutical composition of any one of claims 47-54, wherein:
the hydroxychloroquine, or a pharmaceutically acceptable salt thereof, is hydroxychloroquine sulfate;
the amount of hydroxychloroquine sulfate is 160 mg; and
the pharmaceutical composition comprises about 124 mg hydroxychloroquine free base equivalents.

57. The pharmaceutical composition of any one of claims 47-56, wherein the pH neutralizing agent is sodium bicarbonate.

58. A packaged pharmaceutical product, comprising:
a. a first pharmaceutical composition selected from the group consisting of:
i. a pharmaceutical composition comprising hydroxychloroquine sulfate in an amount selected from 130 mg and 160 mg;
ii. an enterically coated tablet comprising hydroxychloroquine sulfate in an amount selected from 130 mg and 160 mg;
iii. a tablet comprising 100.75 mg or 124 mg hydroxychloroquine free base equivalents in an oil-based matrix;
iv. a pharmaceutical composition comprising hydroxychloroquine sulfate in an amount selected from 130 mg and 160 mg, and sodium bicarbonate; and
c. a controlled release formulation pharmaceutical composition comprising hydroxychloroquine sulfate in an amount selected from 130 mg and 160 mg; and
b. a second pharmaceutical composition that is either protective of retinal health, or beneficial for patients suffering from endometriosis.
